(19) United States
(12) Patent Application Publication
(10) Pub. No.: US 2026/0051364 A1
(43) Pub. Date: Feb. 19, 2026

(54) SYSTEMS AND METHODS FOR GENETIC SCREENING OF EMBRYOS FROM CONSANGUINEOUS PARENTS

(71) Applicant: Herasight Inc., Wilmington, DE (US)

(72) Inventors: Michael CHRISTENSEN, Wilmington, DE (US); Tobias WOLFRAM, Wilmington, DE (US)

(21) Appl. No.: 19/299,016

(22) Filed: Aug. 13, 2025

Related U.S. Application Data
(60) Provisional application No. 63/683,030, filed on Aug. 14, 2024.

(51) Int. Cl. G16B 20/00; G16B 40/20; G16H 50/30

(52) U.S. Cl. CPC G16B 20/00; G16B 40/20; G16H 50/30

(57) ABSTRACT

Described herein are systems and methods and systems for preimplantation genetic testing of an embryo derived from consanguineous parents (PGT-C). The methods involve receiving embryonic genetic data and determining a proportion of the genome in long runs of homozygosity for the entire genome (global F value) as well as for one or more regions of interest within the genome (localized F values). The localized F values are weighted based on one or more factors relating to genetic viability and genetic disorders of the embryo. The global F value and weighted localized F values may be integrated with other genetic data to predict relevant risk scores.

### CROSS-REFERENCE TO RELATED APPLICATIONS

This application claims the benefit of priority to U.S. Provisional Application No. 63/683,030 filed Aug. 14, 2024, the entirety of which is incorporated herein by reference.

### FIELD OF THE DISCLOSURE

Various embodiments of the present disclosure relate generally to the field of genetic screening and preimplantation genetic testing (PGT). More specifically, the present disclosure relates to novel systems and methods for the genetic screening of embryos derived from consanguineous parents (PGT-C), utilizing integrated genetic metrics including global and localized runs of homozygosity (ROH) analysis, polygenic scores, and other genetic data to provide a comprehensive risk assessment.

### BACKGROUND

Consanguineous unions between individuals related as second cousins or closer represent a long-standing practice in numerous communities worldwide, influenced by various sociocultural and economic factors. Such unions, however, are associated with demonstrable elevated health risks for the resulting offspring. The primary biological mechanism underlying these increased health risks is the significantly higher probability of offspring inheriting identical copies of genomic segments from a shared ancestor. This phenomenon leads to an increased frequency and length of continuous stretches of homozygous genotypes that are identical by descent (i.e., runs of homozygosity or ROH). The increased homozygosity elevates the risk that offspring will inherit two copies of deleterious recessive alleles, significantly increasing the incidence of autosomal recessive genetic disorders. Furthermore, studies have indicated an association between consanguinity and an increased risk for various complex diseases and congenital anomalies, including neurodevelopmental disorders, intellectual disability, autism spectrum disorders, cardiovascular disorders, and diabetes mellitus. The impact on public health is particularly pronounced in populations where consanguineous union is common or in populations that have undergone recent genetic bottlenecks (founder effects), leading to reduced overall genetic diversity.

Preimplantation genetic testing (PGT) techniques are utilized to evaluate embryos for genetic viability and risks of disease. Current techniques include PGT for aneuploidy (PGT-A) screens for abnormal chromosome numbers, PGT for monogenic disorders (PGT-M) targets specific known mutations, PGT for structural rearrangements (PGT-SR) identifies chromosomal rearrangements, and PGT for polygenic traits (PGT-P) assesses risk for complex diseases based on the cumulative effect of many variants. However, these techniques are limited in their ability to comprehensively address the unique spectrum of genetic risks associated with consanguinity. They may fail to detect rare variants, novel variants, or cryptic variants whose effects are not fully characterized, which may be notable in the shared ancestry of a consanguineous couple. Further, these techniques often neglect the cumulative impact of multiple recessive variants that individually have small effects but collectively increase risk for complex conditions.

The inherent variation in overall genomic homozygosity observed between sibling embryos from consanguineous parents represents a novel opportunity for embryo selection based directly on the overall burden of consanguinity. Current PGT techniques fundamentally lack a methodology designed to leverage this variation. Furthermore, existing PGT techniques do not systematically integrate this global assessment with a nuanced analysis of localized homozygosity in clinically critical regions, nor combine these factors effectively with other relevant genetic risk data in a manner specifically tailored for comprehensive risk assessment and selection in the context of consanguinity. Consequently, there remains an unmet need for more sophisticated, integrated PGT methods capable of accurately quantifying the specific genetic risks arising from consanguinity, thereby facilitating more informed reproductive decision-making for consanguineous couples utilizing assisted reproductive technologies.

The present disclosure provides, for the first time, systematic methods (PGT-C) and systems for comprehensive risk assessments that enable screening based on quantifiable differences in both global and localized homozygosity burden among embryos from consanguineous parents, representing a significant advancement over merely detecting consanguinity or targeting single gene defects.

### SUMMARY

Provided herein are methods for genetic screening of an embryo. The method may include receiving embryonic genetic data of the embryo; determining a global F value for the embryonic genetic data by applying a global runs of homozygosity (ROH) threshold to the embryonic genetic data; predicting a comprehensive risk score by integrating the global F value; and generating a comprehensive risk score report including the comprehensive risk score. Determining the global F value for the embryonic genetic data may include applying the global ROH threshold to an entire autosomal genome of the embryonic genetic data to identify one or more ROH segments having a size exceeding the global ROH threshold; and dividing a total size of the one or more ROH segments having a size exceeding the global ROH threshold by a total size of the entire autosomal genome of the embryonic genetic data to produce the global F value, wherein each of the one or more ROH segments is a continuous segment of homozygous genotype. The global ROH threshold may range from 1.0 Mb to 200 Mb. Predicting a comprehensive risk score by integrating the global F value may include utilizing a random forest machine learning model, a gradient boosting machine learning model, or a combination thereof.

The embryonic genetic data may include single nucleotide polymorphism chip data or whole genome sequencing data, such as low-coverage whole genome sequencing data. The comprehensive risk score may be indicative of a risk of pregnancy loss or a risk of a genetic disorder.

The method may further include, after determining a global F value: determining a localized F value for a region of interest within the embryonic genetic data by applying a localized ROH threshold to the region of interest; and determining a weighted localized F value by applying one or more weighting factors to the localized F value, wherein predicting the comprehensive risk score may include integrating the global F value and the weighted localized F value. Determining the localized F value for the region of interest within the embryonic genetic data may include: applying a local ROH threshold to the region of interest in the embryonic genetic data to identify one or more ROH segments having a size exceeding the local ROH threshold; and dividing a total size of the one or more ROH segments having a size exceeding the local ROH threshold by a total size of the region of interest in the embryonic genetic data to produce the localized F value, wherein each of the one or more ROH segments may be a continuous segment of homozygous genotype. The local ROH threshold may range from 1.0 Mb to 200 Mb. The local ROH threshold may be less than the global ROH threshold. For example, the global ROH threshold may range from 2.0 Mb to 200 Mb and the local ROH threshold may range from 1.0 Mb to 2.0 Mb. Determining the localized F value for the region of interest within the embryonic genetic data may include determining two or more localized F values for two or more regions of interest within the embryonic genetic data.

Determining the weighted localized F value may include determining one or more weighting factors, each weighting factor being based on size of the region of interest, known clinical significance, pathogenic variant frequency, genetic background or family history, or additional biological information; determining an overall weighting factor based on the one or more weighting factors; and applying the overall weighting factor to the localized F value to produce the weighted localized F value. Known clinical significance may include a measure of clinical significance of homozygosity associated with the region of interest derived from a genetic database storing associations between genomic regions and clinical phenotypes. Pathogenic variant frequency may include a frequency of known pathogenic variants within the region of interest in a reference population. Genetic background or family history may include a known familial genetic risk associated with the region of interest for parents of the embryo. Known familial genetic risk may include a known carrier status for a recessive condition and/or a documented history of a genetic disorder within a family lineage of parents of the embryo. Additional biological information may include gene essentiality, pathway analysis, and/or expression patterns.

The method may further include determining a cumulative localized F value by combining one or more weighted localized F values across one or more regions of interest, wherein determining the comprehensive risk score includes integrating the cumulative localized F value and the global F value.

Further provided herein are systems for genetic screening of an embryo. The system may include: at least one memory storing instructions; and at least one processor configured to execute the instruction to perform operation including receiving embryonic genetic data of the embryo, determining a global F value for the embryonic genetic data by applying a global runs of homozygosity (ROH) threshold to the embryonic genetic data, predicting a comprehensive risk score by integrating the global F value; and generating a comprehensive risk score report including the comprehensive risk score.

Further provided herein are devices for genetic screening of an embryo. The device may include a central processing unit, a communication infrastructure, a main memory, a secondary memory, a communication interface, and an input/output port. The secondary memory may store software instructions and data, including genetic databases and machine learning models. The communication interface may receive genetic data and transmit outputs. The central processing unit may execute instructions stored in the main memory or secondary to perform the steps of the methods described herein

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a diagram illustrating principles of inheritance of identical-by-descent chromosome segments leading to a run of homozygosity in embryos of consanguineous parents, according to techniques discussed herein.

FIG. 2 is a graph illustrating an exemplary statistical distribution of F_ROH values among embryos derived from the consanguineous parents, according to techniques discussed herein.

FIG. 3 is a block diagram depicting an exemplary system for performing PGT-C analysis, according to techniques discussed herein.

FIG. 4A is a flowchart depicting an exemplary method for generating a comprehensive risk score report, according to techniques discussed herein. FIG. 4B is a flowchart depicting further details of step 420 of the exemplary method of FIG. 4A. FIG. 4C is a flowchart depicting further details of step 430 of the exemplary method of FIG. 4A. FIG. 4D is a flowchart depicting further details of step 440 of the exemplary method of FIG. 4A.

FIG. 5 is a block diagram depicting an exemplary device for implementing PGT-C methods, according to techniques discussed herein.

### DETAILED DESCRIPTION

Reference will now be made in detail to the exemplary embodiments of the present disclosure, examples of which may be illustrated in the accompanying drawings.

Wherever possible, the same reference numbers will be used throughout the drawings to refer to the same or like parts.

The methods, devices, and systems disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

As used herein, the term “embryo” refers to a developing organism from the time of fertilization until the end of the eighth week of gestation, including preimplantation stages (e.g., zygote, morula, blastocyst) developed through in vitro fertilization (IVF) or other assisted reproductive technologies.

As used herein, the term “genotyping” refers to the process of determining a genetic variant (allele) an individual possesses at a specific location in their genome.

As used herein, the terms “Run of Homozygosity”, “ROH”, and “ROH segment” refers to a continuous segment of the genome where an individual's two homologous chromosomes carry identical alleles (i.e., exhibit loss of heterozygosity) across a series of consecutive genetic markers. These identical regions typically occur because the parents share a common ancestor, making it more likely that their child inherits the same ancestral DNA segment on both homologous chromosomes (i.e., they are identical by descent, IBD).

As used herein, the term “F” refers to a metric that quantifies features related to the distribution of homozygosity in the genome, including metrics such as F_ROH, which is the proportion of an individual's autosomal genome that resides within ROH segments exceeding a specified minimum length threshold. Length of a segment may be expressed in units of bases or in units of size, such as megabytes (Mb).

As used herein, the terms “Polygenic Score”, “PGS”, “Polygenic Risk Score”, and “PRS” refer to a score calculated for an individual based on the cumulative influence of many genetic variants across their genome, used to estimate their genetic predisposition to a specific trait or disease.

As used herein, the term “rare variant” refers to a genetic variant present at a low frequency (e.g., <1% or <0.1%) in a reference population.

During meiosis (gamete formation), each parent passes one copy of each chromosome to their offspring. The process of meiosis involves chromosomal recombination (crossing over), where genetic material is exchanged between homologous chromosomes before they are segregated into gametes. This recombination process shuffles the specific ancestral segments present on each chromosome. Consequently, each sperm and egg cell produced by an individual parent carries a unique mosaic of segments derived from that individual's own parents (the offspring's grandparents). When two gametes fuse to form an embryo, the specific combination of shuffled parental chromosomes results in a unique genomic constitution for that embryo.

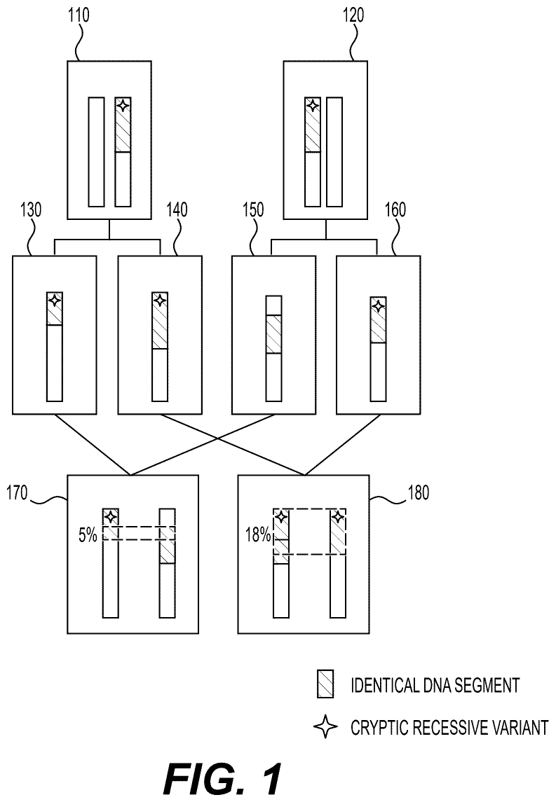

A consanguineous couple is a set of two parents (e.g., father and mother) who are related as second cousins or closer (i.e., having a coefficient of relationship ‘r’≥1/32). Even when embryos are conceived by a consanguineous couple, the stochastic (random) nature of recombination and chromosome segregation during meiosis ensures that the specific IBD segments inherited, and thus the total amount of the genome residing in ROH, will vary substantially among different embryos. FIG. 1 depicts a simplified schematic based on Mendelian inheritance principles to illustrate the origin of Runs of Homozygosity (ROH) stemming from shared ancestry. Father 110 may possess an identical DNA segment containing a cryptic recessive variant to a DNA segment of mother 120. Father 110 may produce sperm cell 130 which possesses a portion of the identical DNA fragment, and the portion may include the cryptic recessive variant. Father 110 may also produce sperm cell 140 which also possesses a portion of the identical DNA fragment, and the portion also includes the cryptic recessive variant. Mother 120 may produce egg cell 150 which possesses a portion of the identical DNA fragment, but the portion does not include the cryptic recessive variant. Mother 120 may also produce egg cell 160, which also possesses a portion of the identical DNA fragment, but the portion does include the cryptic recessive variant. There is a significant probability that the offspring will inherit the identical DNA segment from both father 110 and mother 120. For example, embryo 170 formed from sperm cell 130 and egg cell 150 could possess two portions of the identical gene segment which overlap by about 5%, and one of the two portions could contain the cryptic recessive variant. Embryo 180 formed from sperm cell 140 and egg cell 160 could possess two portions of the identical gene segment which overlap by about 18%, and both portions could contain the cryptic recessive variant.

When this occurs, the resulting homologous chromosomes in the offspring carry DNA sequences in that segment that are not only homozygous (identical in state) but are, more specifically, Identical by Descent (IBD). That is, they are physically identical segments copied from the exact same ancestral source chromosome. Such a contiguous segment of IBD on homologous chromosomes constitutes a Run of Homozygosity (ROH). The length and frequency of these ROH segments are significantly increased in the offspring of consanguineous unions compared to offspring of unrelated individuals, directly reflecting the proportion of the genome inherited IBD from recent common ancestors.

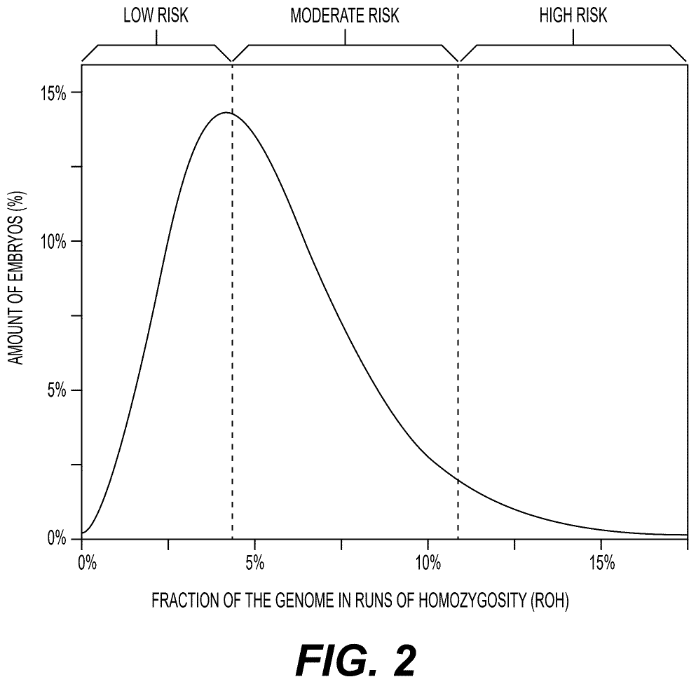

FIG. 2 provides an exemplary statistical distribution of F values calculated across multiple embryos conceived by the same couple (e.g., first cousins). The x-axis represents the F_ROH value (the fraction or percentage of the autosomal genome within ROH segments exceeding a defined length threshold), and the y-axis represents the proportion or frequency of embryos exhibiting that F_ROH value. While the distribution clusters around an average F_ROH value expected for the degree of parental relatedness (e.g., approximately 6.25% for first cousins), the curve clearly demonstrates significant variation: some embryos will, purely by chance, inherit substantially fewer IBD segments and thus exhibit a lower F_ROH, while others will inherit more and have a higher F_ROH.

This inherent, random variation in F among embryos from the same consanguineous couple forms the fundamental basis for the PGT-C methods disclosed herein. By quantifying the global F_ROH (and localized F_ROH in specific regions) for each available embryo, the method allows for the identification and preferential selection of embryos situated at the lower end of the F_ROH distribution. A lower F_ROH value directly correlates with a reduced probability that the embryo has inherited two copies of any deleterious recessive allele (whether known or unknown/cryptic) located within homozygous segments derived from shared ancestors. Therefore, selecting embryos with lower F_ROH provides a mechanism to significantly mitigate the elevated genetic risks associated with consanguinity. As shown in FIG. 2, embryos can be categorized into risk strata (e.g., low, moderate, high risk) based on their calculated F_ROH relative to the distribution observed for that specific couple or relatedness level, facilitating informed decision-making for embryo transfer.

Provided herein are systems and methods for preimplantation genetic testing of embryos derived from consanguineous parents (PGT-C), addressing the unmet need for selection strategies based on the variable levels of genomic homozygosity inherent in such embryos. This approach integrates multiple genetic metrics and employs advanced data processing techniques to generate a comprehensive risk assessment tailored to the unique genetic landscape of these embryos, aiming to reduce the risk of inheriting two copies of damaging recessive variants, including those not detected by standard carrier screening and therefore offering significant advantages over existing screening methodologies.

In some aspects, a method for genetic screening of embryos from consanguineous parents may comprise: obtaining genetic data from at least one embryo conceived by said consanguineous parents; calculating, using a processor, a global F value for the at least one embryo, wherein F represents a metric that quantifies aspects related to homozygosity in the genome, such as the proportion of the embryo's genome in runs of homozygosity (F_ROH) above a first predetermined length threshold; identifying one or more genes or genomic regions of interest based on information from at least one genetic database; calculating, using the processor, one or more localized F values for the identified genes or genomic regions of interest for the at least one embryo, wherein the localized F calculation may use a second predetermined ROH length threshold different from the first; applying, using the processor, a multi-factor weighting system to the one or more localized F values, said weighting system considering factors related to the genetic and clinical significance of homozygosity in said regions; and generating, using the processor, a comprehensive risk score for the at least one embryo based at least in part on the global F value and the weighted localized F values. This dual analysis uniquely allows for selection based on overall consanguinity burden (via global F variation between embryos) and further refinement based on risks within specific genomic regions (via weighted localized F).

In some embodiments, the method may further comprise calculating polygenic scores (PGS) for predetermined conditions for the at least one embryo and incorporating said PGS into the comprehensive risk score. In some embodiments, the method may involve identifying rare genetic variants within the embryo's genetic data and incorporating their potential impact into the risk score. In some embodiments, the method may incorporate genetic factors (e.g., using metrics like S_HET) to refine the risk assessment, potentially based on the parents' ancestry. Genetic factors are genetic characteristics, such as allele frequencies or linkage disequilibrium patterns, that may be particular to certain ethnic or geographically defined populations. Metrics like S_HET (Selection against Heterozygotes) may reflect such factors.

In some embodiments, the method may incorporate a targeted analysis based on known familial genetic risks, wherein specific genes or genomic regions associated with a condition present in the family are prioritized, potentially involving adjustment of ROH length thresholds or weighting factors for those specific regions. In some embodiments, the comprehensive risk score is generated using a machine learning algorithm, such as a random forest model, a gradient boosting model. In some embodiments, potential disease risks contributing to the comprehensive risk score are weighted according to Disability-Adjusted Life Years (DALYs) or similar metrics of severity.

Advantageously, the disclosed systems and methods are designed to be compatible with various types of genetic data, including, but not limited to, dense single nucleotide polymorphism (SNP) chip data, whole genome sequencing (WGS) data, and low-coverage or ultra-low coverage sequencing data (potentially enhanced using parental WGS data). The method facilitates the selection of embryos with lower predicted genetic risk for transfer during in vitro fertilization (IVF) procedures.

Further provided herein are systems, computer-implemented systems, and devices for implementing or performing the methods described herein.

### System Architecture

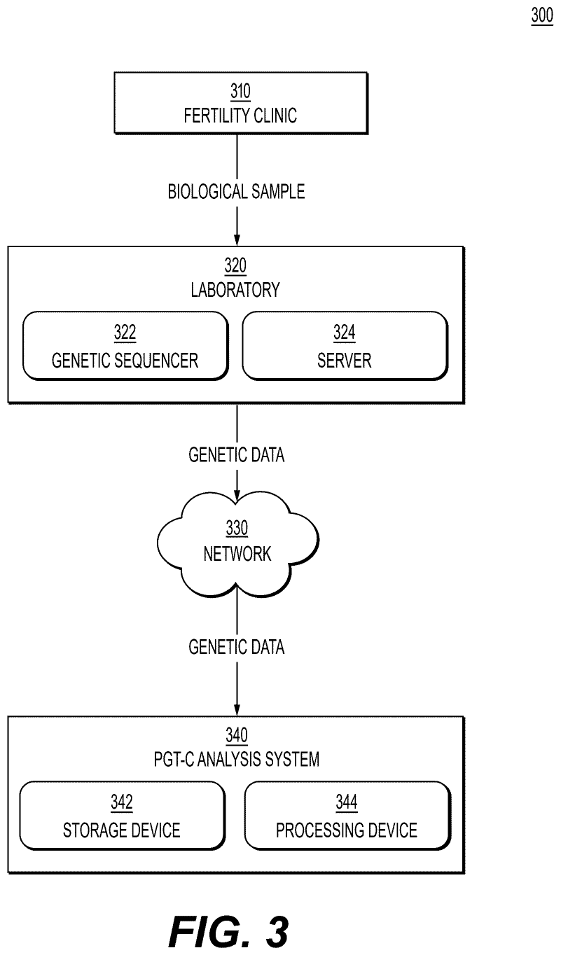

FIG. 3 illustrates an exemplary system 300 for performing PGT-C. Consanguineous parents may undergo an IVF procedure at fertility clinic 310 to produce an embryo. The embryo may be biopsied to produce a biological sample (e.g., trophectoderm cells), which may be provided to laboratory 320 for processing. A biological sample (e.g., blood or saliva) may be obtained from one or both of the consanguineous parents at fertility clinic 310, and may also be provided to laboratory 320 for processing. Laboratory 320 may include a genetic sequencer 322 and a server 324. Laboratory 320 may use genetic sequencer 322 to produce genetic data from the embryonic biological sample and/or the parental biological sample. Laboratory 320 may use server 324 to provide genetic data via network 330 (e.g., a secure internet connection) to PGT-C analysis system 340. PGT-C analysis system 340 may include a memory 342 and a processor 344, and may be located at laboratory 320 or at a separate data processing entity. Memory 342 may store instructions which can be executed by processor 344 to perform PGT-C methods as described herein. For example, PGT-C analysis system 340 may calculate a global F, identify a region of interest, calculate a weighted localized F, and/or integrate other metrics (PGS, rare variants, etc.) to generate a comprehensive risk score report for the embryo. The comprehensive risk score report may be transmitted via network 330 to fertility clinic 310 and/or a genetic counselor to inform decisions regarding embryo selection for transfer.

### Methods for PGT-C

The PGT-C methods described herein recognize and exploit natural variation in global F levels among sibling embryos produced by the same consanguineous couple. Due to the random nature of meiotic recombination, different embryos inherit different combinations of ancestral chromosome segments, leading to quantifiable differences in their overall homozygosity burden. PGT-C provides a systematic means to measure this variation (via global F) and utilize it as a primary criterion for embryo selection, aiming to choose embryos with a lower overall consanguinity impact. This foundational assessment may then be further refined by detailed analysis of localized F in critical regions, combined with other genetic risk factors, providing a multi-layered approach unavailable in prior art.

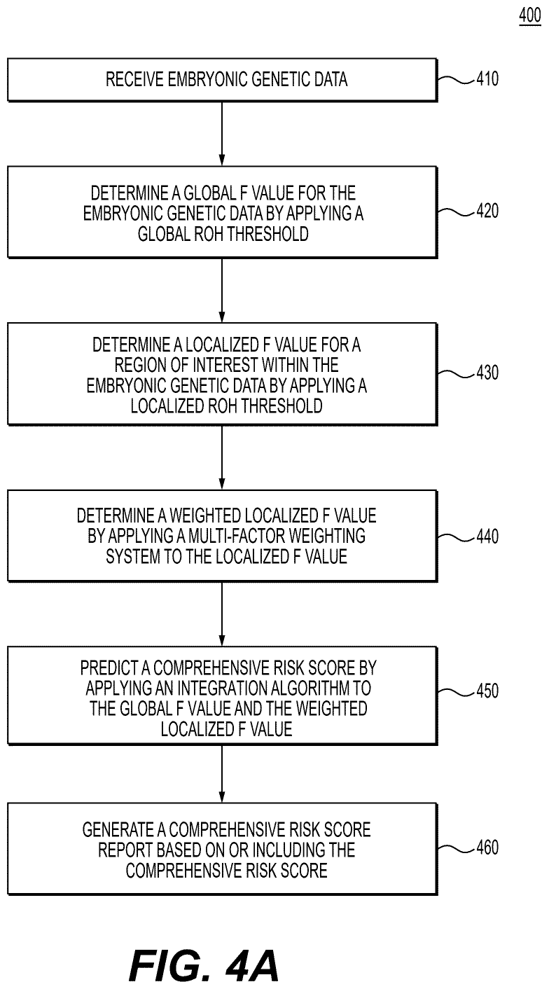

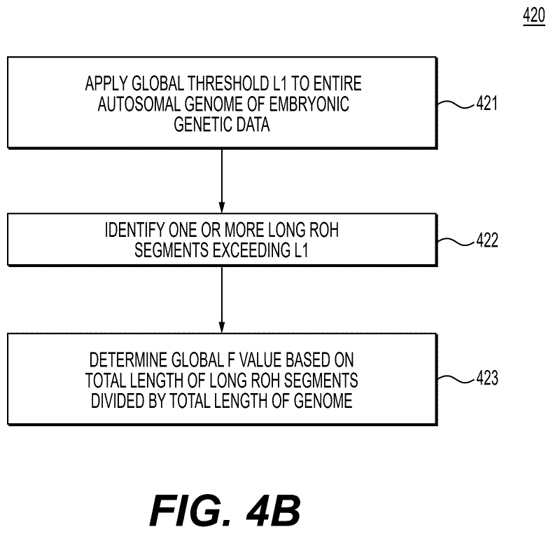

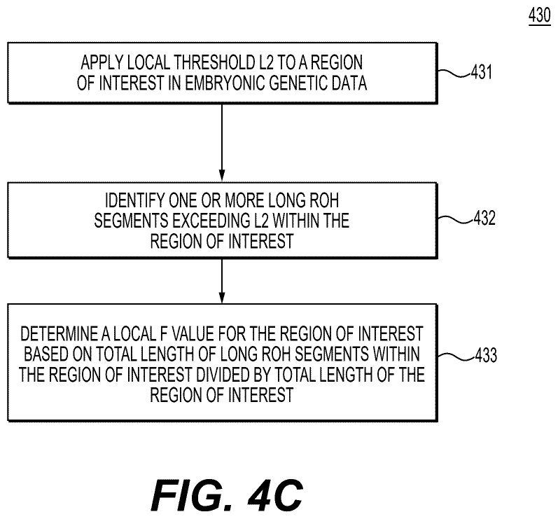

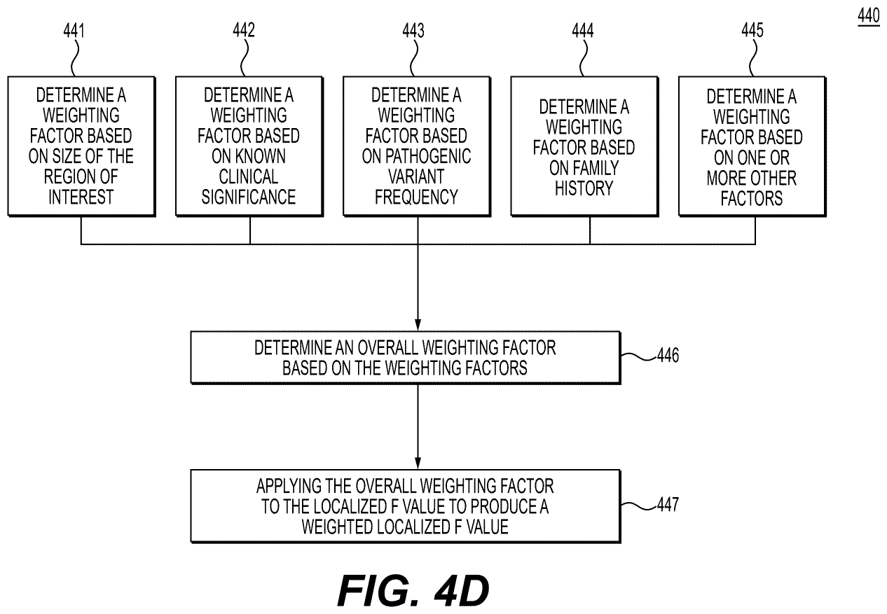

FIG. 4A depicts a flowchart illustrating an exemplary method 400 for PGT-C. At step 410, method 400 may include receiving genetic data for an embryo derived from consanguineous parents. The embryonic genetic data may be produced by a genetic sequencer analyzing a biological sample from the embryo. For example, an embryo may be produced by performing an IVF procedure and culturing an embryo derived from two consanguineous parents. A biopsy, such as a trophectoderm biopsy at day 5 or 6, may be taken to produce the biological sample (e.g., trophectoderm cells). If the amount of genetic material within the biological sample is very low, whole genome amplification (WGA) may be performed to increase the amount of genetic material for subsequent genotyping or sequencing to produce embryonic genetic data for analysis. The genetic data may be of various types, including dense single nucleotide polymorphism (SNP) array data, whole genome sequencing (WGS) data, or low-or ultra-low coverage WGS data. In embodiments using low-coverage embryo data, accuracy may be enhanced by utilizing higher-coverage parental WGS data, for example, using methods to improve imputation or phasing based on parental haplotypes, for example, using methods to improve imputation or phasing based on parental haplotypes. Such methods for enhancing the accuracy of embryo genetic data, including for generating a predicted genome from low-coverage data, are described in U.S. Patent Application Publication No. 2025/0034645, which is incorporated herein by reference in its entirety.

At step 420, method 400 may include determining a global F value for the embryo based on the embryonic genetic data. Determining the global F value may include computationally scanning the autosomal genome of the embryo to identify one or more ROH segments having a length and/or size exceeding a first predetermined length threshold, which may be referred to as a global ROH threshold. The global ROH threshold may range from 1.0 Mb to 200 Mb, from 1.0 Mb to 150 Mb, from 1.0 Mb to 100 Mb, from 1.0 Mb to 50 Mb, from 1.0 Mb to 20 Mb, from 1.0 Mb to 10 Mb, from 1.0 Mb to 9.0 Mb, from 1.0 Mb to 8.0 Mb, from 1.0 Mb to 7.0 Mb, from 1.0 Mb to 6.0 Mb, from 1.0 Mb to 5.0 Mb, from 1.0 Mb to 4.0 Mb, or from 1.0 Mb to 3.0 Mb. For example, the global ROH threshold may be about 1.0 Mb, about 1.1 Mb, about 1.2 Mb, about 1.3 Mb, about 1.4 Mb, about 1.5 Mb, about 1.6 Mb, about 1.7 Mb, about 1.8 Mb, about 1.9 Mb, about 2.0 Mb, about 2.1 Mb, about 2.2 Mb, about 2.3 Mb, about 2.4 Mb, about 2.5 Mb, about 2.6 Mb, about 2.7 Mb, about 2.8 Mb, about 2.9 Mb, or about 3.0 Mb. The global ROH threshold may be greater than 3.0 Mb, such as about 3.1 Mb, about 3.2 Mb, about 3.3 Mb, about 3.4 Mb, about 3.5 Mb, about 3.6 Mb, about 3.7 Mb, about 3.8 Mb, about 3.9 Mb, about 4.0 Mb, about 4.1 Mb, about 4.2 Mb, about 4.3 Mb, about 4.4 Mb, about 4.5 Mb, about 4.6 Mb, about 4.7 Mb, about 4.8 Mb, about 4.9 Mb, about 5.0 Mb, about 10 Mb, about 15 Mb, about 20 Mb, about 30 Mb, about 40 Mb, about 50 Mb, about 60 Mb, about 70 Mb, about 80 Mb, about 90 Mb, about 100 Mb, about 110 Mb, about 120 Mb, about 130 Mb, about 140 Mb, about 150 Mb, about 160 Mb, about 170 Mb, about 180 Mb, about 190 Mb, about 200 Mb, or any value therebetween. Algorithms such as PLINK or similar tools using sliding windows or hidden Markov models (HMMs) may be employed. The total length of these qualifying ROH segments may then be summed and divided by the total analyzed autosomal genome length to yield the global F value.

An embodiment of step 420 is depicted in FIG. 4B. At step 421, a global threshold L1 (e.g., L1=2.5 Mb) may be applied to the entire autosomal genome of the embryonic genetic data. At step 422, one or more ROH segments having a length and/or size exceeding global threshold L1 may be identified as long ROH segments. At step 423, the global F value may be determined by dividing a total length of the long ROH segments identified in step 422 by a total length of the genome of the embryonic genetic data.

Returning now to FIG. 4A, at step 430, method 400 may optionally include determining a localized F value for a region of interest within the embryonic genetic data, such as a gene. The region of interest may be identified using information curated in one or more genetic databases. A genetic database is a curated repository of information linking genes and genetic variants to phenotypes, diseases, and biological functions. Examples of such genetic databases include ClinVar (for clinically relevant variants/genes), OMIM (for Mendelian disorders, particularly autosomal recessive conditions relevant to consanguinity), and GWAS catalogs (for regions associated with complex traits or diseases where homozygosity might modulate risk). Further examples include Ensembl and the UCSC Genome Browser. The region of interest may be selected based on a known association with a severe recessive disorder, a developmental pathway, or a condition prevalent in the specific population background of the parents. Determining the localized F value for the region of interest may mirror determining the global F value but restricted to specific coordinates for the region of interest within the embryonic genetic data. A second predetermined ROH length threshold, referred to as a local ROH threshold, may be applied for determining the localized F value. The local ROH threshold can be set lower than the global ROH threshold to capture potentially significant homozygosity within a gene that might not meet the global ROH length criteria. The local ROH threshold may range from 1.0 Mb to 2.0 Mb. For example, the local ROH threshold may be about 1.0 Mb, about 1.1 Mb, about 1.2 Mb, about 1.3 Mb, about 1.4 Mb, about 1.5 Mb, about 1.6 Mb, about 1.7 Mb, about 1.8 Mb, about 1.9 Mb, or about 2.0 Mb.

An embodiment of step 430 is depicted in FIG. 4C. At step 431, a local threshold L2 may be applied to a region of interest in the embryonic genetic data. Local threshold L2 may be a smaller length (e.g., L2=1.5 Mb) than global threshold L1 but longer than a potentially lower localized threshold. At step 432, one or more ROH segments within the region of interest having lengths exceeding L2 may be identified as long ROH segment within the region of interest. At step 433, a local F value may be determined for the region of interest by dividing a total length of the long ROH segments in the region of interest by a total length of the region of interest.

Returning to FIG. 4A, at step 440, method 400 may optionally include determining a weighted localized F value by applying a multi-factor weighting system to the localized F value. This step may assign a significance score to homozygosity in a region of interest based on predefined criteria. For region of interest i, a weight W_i may be determined based on one or more factors. Size of the region of interest may be a factor, as a larger sized region may have a higher probability of harboring deleterious variants. Known clinical significance may be another factor, as a region containing genes where homozygosity or loss-of-function is known to cause severe autosomal recessive disorders (e.g., from OMIM/ClinVar) may receive a higher weight. Pathogenic variant frequency may be another factor, as a region known to harbor a pathogenic variant that is rare in the general population but enriched in the family/population may receive a higher weight if found homozygous. Genetic background and/or family history of the parents may be another factor. If the parents are known carriers for a specific recessive condition, or if a specific recessive disorder is present in the family (even without a known causative SNP), the corresponding gene or region of interest may receive a significantly higher weight. This may constitute a targeted analysis which may involve adjusting the ROH length threshold specifically for these familial risk regions to capture potentially smaller IBD segments relevant to the known family risk. Weighting can also consider regions shared identically (IBD) with affected family members (e.g., a previously affected sibling) if such data is available. Additional biological information, such as gene essentiality, pathway analysis, or expression patterns, may be other factors. The weighting factors may be combined computationally to produce the final weight W_i for each localized F_i.

An embodiment of step 440 is depicted in FIG. 4D. For each region i, a weighting factor W_i may be determined based on size of the region of interest 441. Larger regions might have a higher prior probability of harboring deleterious variants). For each region i, a weighting factor W_i may be determined based on known clinical significance 442. Regions containing genes where homozygosity or loss-of-function is known to cause severe autosomal recessive disorders (e.g., from OMIM/ClinVar) receive higher weights). For each region i, a weighting factor W_i may be determined based on pathogenic variant frequency 443. Regions known to harbor pathogenic variants that are rare in the general population but might be enriched in the family or population may receive higher weight if found homozygous. For each region i, a weighting factor W_i may be determined based on a couple's genetic background/family history 444. If the parents are known carriers for specific recessive conditions, or if a specific recessive disorder is present in the family even without a known causative SNP, the corresponding gene or region may receive a significantly higher weight. For each region i, a weighting factor W_i may be determined based on other factors 445, such as gene essentiality, pathway analysis, or expression patterns. Determining a weight W_i based on any one of these factors may include a targeted analysis for the factor. For example, a targeted analysis of the couple's genetic background/family history may include adjusting the ROH length threshold specifically for these familial risk regions to capture potentially smaller IBD segments relevant to the known family risk. The targeted analysis may include considering regions shared identically (IBD) with affected family members (e.g., a previously affected sibling), if such data is available. At step 446, an overall weighting factor may be determined based on one or more of these weighting factors. At step 447, the overall weighting factor may be applied to the localized F value to produce the weighted F value.

The method may optionally include determining a cumulative weighted localized F score. For example, a cumulative weighted localized F score may be determined as the sum of (localized F_i*W_i) across all regions i. This cumulative score provides a single metric representing the overall burden of homozygosity in clinically relevant genomic areas, adjusted for significance. This weighted score (or the individual weighted values) may then be used in generating the comprehensive risk score.

Returning to FIG. 4A, at step 450, a comprehensive risk score may be predicted for the embryo. The comprehensive risk score may integrate information from the global F value (reflecting overall inbreeding). Optionally, the comprehensive risk score may further integrate a weighted localized F value (reflecting risk in a specific critical region), a cumulative localized F score, and/or any other integrated metrics (PGS, rare variants, population factors, etc.). The integration may be performed using various algorithms, ranging from a simple weighted average to more complex machine learning models trained on relevant datasets. In some techniques, the integration may be performed by a random forest model, a gradient boosting model, a support vector machine model, a neural network model, a hidden Markov model, or a combination thereof. In some techniques, the integration may be performed using a random forest model. A random forest model is an ensemble machine learning model that combines the outputs of multiple decision trees (e.g., classifier models) to reach a single result. A single decision tree (e.g., classifier model) may be prone to problems such as bias and overfitting. The random forest model utilizes feature randomness (also known as feature bagging or the random subspace method) to generate a random subset of features which ensures low correlation among the individual decision trees. By utilizing multiple decision trees with random subsets of features and low correlation to each other, the random model may produce more accurate predictions than a single classifier model. In some techniques, the integration may be performed using a gradient boosting model. A gradient boosting model is an ensemble machine learning model that also utilizes multiple decision trees. Unlike a random forest model, a gradient boosting model utilizes boosting to iteratively train its models to correct previous mistakes by minimizing a loss function. In some techniques, integration may performed using a combination of a random forest model and a gradient boosting model. The machine learning model may be trained on input features such as the global F value, the one or more weighted localized F values, and the one or more polygenic scores calculated for a training set of embryos with known clinical outcomes.

Where such machine learning algorithms are employed for generating the comprehensive risk score, they offer the advantage of capturing complex, non-linear interactions between the various input metrics (global F, weighted localized F scores, PGS, rare variant impacts, etc.) that may be missed by simpler linear models or weighted averages. Training such models may involve utilizing datasets comprising genetic data from embryos or individuals with known clinical outcomes (e.g., live birth, diagnosis of genetic disorders, developmental milestones). Feature selection may focus on the calculated genetic metrics described herein. Model performance may be validated using standard techniques such as k-fold cross-validation on the training data and, may be tested on independent validation cohorts to ensure generalizability and robustness before clinical application. In some embodiments, the contribution of different potential disease risks to the score may be weighted according to their severity and impact, for instance, by using disability-adjusted life years (DALYs) associated with the conditions. DALYs are a measure of overall disease burden, expressed as the number of years lost due to ill-health, disability, or early death.

Other relevant genetic metrics may include calculating Polygenic Scores (PGS) for predetermined complex diseases or traits relevant to the family history or population background, identifying and assessing the potential impact of rare variants, particularly homozygous rare variants within ROH segments or in disease-associated genes, and incorporating population-specific genetic factors, potentially using metrics derived from population databases or measures like S_HET.

At step 460, the comprehensive risk score and supporting information may be outputted, typically in a report format. This report may aid a clinician or genetic counselor in discussing the relative genetic risks associated with each embryo and facilitates informed decision-making regarding embryo selection for transfer, aiming to reduce the likelihood of conceiving a child affected by severe genetic disorders associated with consanguinity or increased risk of pregnancy loss due to e.g. miscarriage or stillbirth.

In some embodiments, a method for genetic screening of an embryo may comprise: receiving embryo genetic data from at least one embryo; calculating, using a computer processor communicatively coupled to a memory storing the embryo genetic data, a global F value for the at least one embryo, wherein the global F value is calculated as the proportion of the embryo's autosomal genome residing in runs of homozygosity (ROH), defined as continuous segments of homozygous genotypes exceeding a first predetermined length threshold; identifying, using the computer processor, one or more genomic regions of interest by querying at least one genetic database storing associations between genomic regions and clinical phenotypes; calculating, using the computer processor, one or more localized F values for the identified one or more genomic regions of interest for the at least one embryo based on the embryo genetic data, wherein said localized F values quantify homozygosity within said regions of interest exceeding a second predetermined length threshold; applying, using the computer processor, a multi-factor weighting system to the one or more calculated localized F values to produce one or more weighted localized F values, wherein the weighting system assigns weights based on factors comprising at least: (i) a measure of clinical significance of homozygosity associated with the respective genomic region of interest derived from said at least one genetic database, and (ii) known familial genetic risks associated with the respective genomic region for parents of the at least one embryo; and generating, using the computer processor, a comprehensive risk score for the at least one embryo, wherein the comprehensive risk score is based at least in part on the calculated global F value and the one or more weighted localized F values.

In some embodiments, the comprehensive risk score is indicative of a genetic risk associated with consanguinity to facilitate selection of an embryo for transfer in an IVF procedure. In some embodiments, the comprehensive risk score is indicative of a risk of pregnancy loss due to e.g. still birth and miscarriage for the at least one embryo.

In some embodiments, the embryo genetic data is selected from the group consisting of: dense single nucleotide polymorphism (SNP) chip data, whole genome sequencing (WGS) data, and low-coverage WGS data.

In some embodiments, the embryo genetic data is low-coverage WGS data, the method further comprises enhancing the embryo genetic data using parental WGS data prior to calculating the global F value and the one or more localized F values.

In some embodiments, the first predetermined length threshold is selected from a range of 2.0 Mb to 3.0 Mb. In some embodiments, the first predetermined length threshold is 2.5 Mb. In some embodiments, the second predetermined ROH length threshold is different from the first predetermined length threshold. In some embodiments, the second predetermined ROH length threshold is lower than the first predetermined length threshold. In some embodiments, the second predetermined ROH length threshold is selected from a range of 1.0 Mb to 2.0 Mb. In some embodiments, the second predetermined ROH length threshold is 1.5 Mb.

In some embodiments, the at least one genetic database is selected from the group consisting of: ClinVar, Online Mendelian Inheritance in Man (OMIM), and a Genome-Wide Association Study (GWAS) catalog.

In some embodiments, the factors considered by the multi-factor weighting system further comprise one or more selected from the group consisting of: size of the genomic region of interest, and frequency of known pathogenic variants within the region in a reference population. In some embodiments, known familial genetic risks include known carrier status for specific recessive conditions or a documented history of a specific genetic disorder within the family lineage of parents of the at least one embryo.

In some embodiments, the method further comprises performing a targeted analysis for the known familial genetic risks, wherein the targeted analysis comprises: prioritizing the genomic regions associated with the known familial genetic risk in the localized F calculations; and applying increased weighting, via the multi-factor weighting system, to the localized F values calculated for said prioritized genomic regions. In some embodiments, the targeted analysis further comprises adjusting the second predetermined ROH length threshold specifically for the prioritized genomic regions.

In some embodiments, the method further comprises calculating, using the computer processor, a cumulative localized F score by combining the one or more weighted localized F values across the one or more genomic regions of interest, wherein the comprehensive risk score is based at least in part on the cumulative localized F score.

In some embodiments, the method further comprises calculating, using the computer processor, one or more polygenic scores (PGS) for predetermined conditions for the at least one embryo based on the embryo genetic data; and incorporating the one or more calculated PGS into the generation of the comprehensive risk score.

In some embodiments, the method further comprises: identifying, using the computer processor, one or more rare variants within the embryo genetic data; and incorporating into the comprehensive risk score an assessment of potential impact of said rare variants, wherein the assessment utilizes a variant pathogenicity prediction algorithm or assigns a classification based on ACMG/AMP variant interpretation guidelines.

In some embodiments, generating the comprehensive risk score further comprises incorporating population-specific genetic factors derived from ancestry information of parents of the at least one embryo.

In some embodiments, generating the comprehensive risk score comprises utilizing a machine learning algorithm. In some embodiments, the machine learning algorithm is selected from the group consisting of: a random forest model, a gradient boosting model, a support vector machine model, a neural network model, and a hidden Markov model. In some embodiments, the machine learning algorithm is trained using input features comprising the global F value, the one or more weighted localized F values, and/or one or more polygenic scores calculated for a training set of embryos with known clinical outcomes.

In some embodiments, the method further comprises weighting potential disease risks contributing to the comprehensive risk score according to Disability-Adjusted Life Years (DALYs) associated with respective diseases linked to the identified genomic regions of interest or polygenic scores.

In some embodiments, the method further comprises selecting, based on comparing the comprehensive risk score generated for the at least one embryo with comprehensive risk scores generated for one or more other embryos conceived by the same parents, an embryo with a lower comprehensive risk score for transfer in an in vitro fertilization procedure.

### Systems and Devices for PGT-C

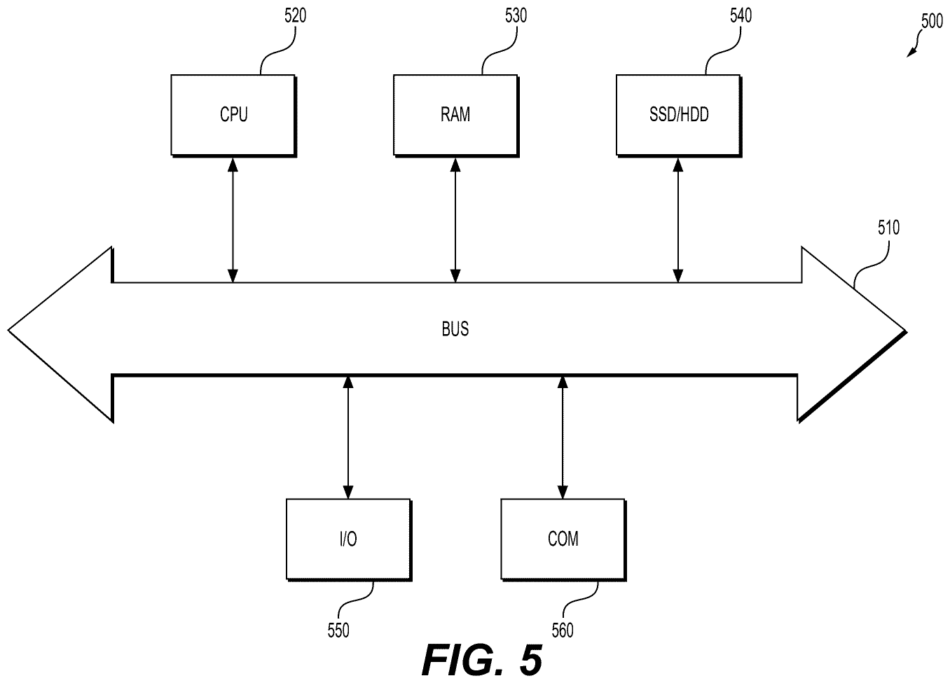

FIG. 5 illustrates an exemplary computer-implemented system or device 500 that may execute the PGT-C techniques presented herein, corresponding to the PGT-C analysis system 340 in FIG. 3. Device 500 includes a central processing unit (CPU) 520, which may be a single or multi-core processor. CPU 520 is connected to a communication infrastructure 510 (e.g., bus, network). Device 500 also includes main memory 540 (e.g., RAM) and may include secondary memory 530 (e.g., hard disk, solid-state drive). Secondary memory 530 may store software instructions and data, including genetic databases and machine learning models. Device 500 includes a communication interface (COM) 560 for receiving genetic data and transmitting results, potentially over network 330. Input/output ports 550 may connect to peripherals like monitors and keyboards. CPU 520 may execute software instructions stored in memory (540, 530) to perform the steps of the PGT-C methods described herein, including calculating F, applying weighting, integrating metrics, and generating risk scores. The system may be implemented on a single computer, a server, or distributed across multiple computing devices (e.g., in a cloud computing environment).

### Additional Applications

Beyond the application of selecting embryos with lower genetic risk for transfer in IVF cycles involving consanguineous couples, the methods and systems described herein have potential utility in other areas.

The methods and systems described herein may be used to predict risk of pregnancy loss. Elevated levels of homozygosity and other genetic risk factors including polygenic scores and rare variant burden scores, particularly in certain genomic regions, may be associated with increased risk of pregnancy loss due to miscarriage and stillbirth. The comprehensive risk score could incorporate this aspect.

The methods and systems described herein may be used for the purposes of genetic counseling. The detailed risk assessment provided by PGT-C can serve as a valuable tool for genetic counselors advising consanguineous couples about their reproductive risks and options.

The methods and systems described herein may be used in post-natal screening. The principles of calculating global and weighted localized F could be applied to genetic data from born individuals (children or adults) from consanguineous backgrounds to identify those at potentially higher risk for late-onset recessive disorders, complex diseases influenced by homozygosity, or neurodevelopmental outcomes like intellectual disability, allowing for earlier monitoring or intervention.

The methods and systems described herein may be used in conducting population health studies or other research applications. Applying these methods to large datasets from populations with high rates of consanguinity can provide insights into the population-level burden of recessive diseases and the effects of inbreeding on complex traits. The methodology can be used in research settings to investigate the specific impact of homozygosity in different genomic regions on various phenotypes and disease risks.

As understanding of genomic function improves, the weighting system could become more sophisticated. In the future, F analysis might even inform selection strategies at the chromosome level, although this remains speculative.

### EXAMPLES

### Example 1 (Prophetic): Predicting Consanguinity Burden for IVF-Derived Blastocysts

Consanguineous parents (first degree cousins) undergo IVF to produce five blastocysts available for testing: Embryo 1, Embryo 2, Embryo 3, Embryo 4, and Embryo 5. Standard PGT-A reveals all are euploid. Following standard statistical genetic modeling and without further background endogamy in the population, in expectation, their embryos will have an F_ROH value of 6.25%. Applying the PGT-C method: Embryo 1 shows a global F_ROH of 10.5%; Embryo 2:4.8%; Embryo 3:2.1%; Embryo 4:7.0%; Embryo 5:3.3%. Based on global F_ROH variation, Embryos 2, 3, and 5 present a lower overall consanguinity burdens than Embryos 1 and 4.

### Example 2 (Prophetic): Predicting Lowest Overall Genetic Risk Related to Consanguinity

Further analysis of Embryos 2, 3, and 5 from Example 1 reveals that Embryo 2 has significant weighted localized F_ROH in a region linked to a severe recessive metabolic disorder known in the family, despite its moderate global F_ROH. Embryo 3, however, has the lowest global F_ROH and also low weighted localized F_ROH scores in critical regions, along with acceptable polygenic scores for relevant complex diseases. Embryo 5 has low global F_ROH but high localized F_ROH in a region associated with neurodevelopmental delay. In this scenario, PGT-C provides multi-layered data enabling the selection of Embryo 3 as having the lowest predicted overall genetic risk related to consanguinity, a decision not possible with standard PGT alone.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

1. A method for genetic screening of an embryo, the method comprising:
receiving embryonic genetic data of the embryo;
determining a global F value for the embryonic genetic data by applying a global runs of homozygosity (ROH) threshold to the embryonic genetic data;
predicting a comprehensive risk score by integrating the global F value; and
generating a comprehensive risk score report including the comprehensive risk score.

2. The method of claim 1, wherein determining the global F value for the embryonic genetic data comprises:
applying the global ROH threshold to an entire autosomal genome of the embryonic genetic data to identify one or more ROH segments having a size exceeding the global ROH threshold; and
dividing a total size of the one or more ROH segments having a size exceeding the global ROH threshold by a total size of the entire autosomal genome of the embryonic genetic data to produce the global F value,
wherein each of the one or more ROH segments is a continuous segment of homozygous genotype.

3. The method of claim 2, wherein the global ROH threshold ranges from 1.0 Mb to 200 Mb.

4. The method of claim 1, wherein predicting a comprehensive risk score by integrating the global F value comprises utilizing a random forest machine learning model, a gradient boosting machine learning model, or a combination thereof.

5. The method of claim 1, wherein the embryonic genetic data is single nucleotide polymorphism chip data.

6. The method of claim 1, wherein the embryonic genetic data is whole genome sequencing data.

7. The method of claim 1, wherein the embryonic genetic data is low-coverage whole genome sequencing data.

8. The method of claim 1, wherein the comprehensive risk score is indicative of a risk of pregnancy loss or a risk of a genetic disorder.

9. The method of claim 1, further comprising, after determining a global F value:
determining a localized F value for a region of interest within the embryonic genetic data by applying a localized ROH threshold to the region of interest; and
determining a weighted localized F value by applying one or more weighting factors to the localized F value,
wherein predicting the comprehensive risk score comprises integrating the global F value and the weighted localized F value.

10. The method of claim 9, wherein determining the localized F value for the region of interest within the embryonic genetic data comprises:
applying a local ROH threshold to the region of interest in the embryonic genetic data to identify one or more ROH segments having a size exceeding the local ROH threshold; and
dividing a total size of the one or more ROH segments having a size exceeding the local ROH threshold by a total size of the region of interest in the embryonic genetic data to produce the localized F value,
wherein each of the one or more ROH segments is a continuous segment of homozygous genotype.

11. The method of claim 10, wherein the local ROH threshold is less than the global ROH threshold.

12. The method of claim 9, wherein determining the localized F value for the region of interest within the embryonic genetic data comprises determining two or more localized F values for two or more regions of interest within the embryonic genetic data.

13. The method of claim 9, wherein determining the weighted localized F value comprises:
determining one or more weighting factors, each weighting factor being based on size of the region of interest, known clinical significance, pathogenic variant frequency, genetic background or family history, or additional biological information;
determining an overall weighting factor based on the one or more weighting factors; and
applying the overall weighting factor to the localized F value to produce the weighted localized F value.

14. The method of claim 13, wherein the known clinical significance includes a measure of clinical significance of homozygosity associated with the region of interest derived from a genetic database storing associations between genomic regions and clinical phenotypes.

15. The method of claim 13, wherein the pathogenic variant frequency includes a frequency of known pathogenic variants within the region of interest in a reference population.

16. The method of claim 13, wherein the genetic background or family history includes a known familial genetic risk associated with the region of interest for parents of the embryo.

17. The method of claim 13, wherein the additional biological information includes as gene essentiality, pathway analysis, and/or expression patterns.

18. The method of claim 1, further comprising determining a cumulative localized F value by combining one or more weighted localized F values across one or more regions of interest, wherein determining the comprehensive risk score includes integrating the cumulative localized F value and the global F value.

19. A system for genetic screening of an embryo, the system comprising:
at least one memory storing instructions; and
at least one processor configured to execute the instruction to perform operation comprising:
receiving embryonic genetic data of the embryo;
determining a global F value for the embryonic genetic data by applying a global runs of homozygosity (ROH) threshold to the embryonic genetic data;
predicting a comprehensive risk score by integrating the global F value; and
generating a comprehensive risk score report including the comprehensive risk score.
